(19) United States
(12) Patent Application Publication
(10) Pub. No.: US 2011/0230343 A1
(43) Pub. Date: Sep. 22, 2011

(54) Method for the Manufacture of Microparticles Comprising an Effect Substance

(75) Inventors: Michael Schroers, Bad Duerkheim (DE); Rainer Dyllick-Brenzinger, Neustadt (DE); Michael Merk, Arese (IT); Heiko Barg, Weinheim (DE)

(73) Assignee: BASF SE, Ludwigshafen (DE)

(21) Appl. No.: 13/125,388

(22) PCT Filed: Oct. 14, 2009
(86) PCT No.: PCT/EP09/63379

§ 371 Date: Apr. 21, 2011

(30) Foreign Application Priority Data
Oct. 24, 2008 (EP) 08167474.9

(51) Int. Cl. A01C 1/06; C12P 7/62; C12P 13/00; C12P 17/02; A01N 25/28; A01P 3/00; A01P 7/02; A01P 7/04; A01P 13/00; A23K 1/16; A23L 1/30

(52) U.S. Cl. 504/100; 435/135; 435/128; 435/123; 504/359; 424/497; 426/96

(57) ABSTRACT

A subject matter of the present invention is a process for the preparation of effect compound-comprising microparticles M comprising A) the formation of a crude suspension of microparticles A by means of enzymatic polyester synthesis in an inverse miniemulsion comprising enzyme, effect compound and polyester monomers; and B) the polymerization of wall monomers from the group consisting of ethylenically unsaturated monomers, polyisocyanates and polyepoxides in the crude suspension of microparticles A. Furthermore, the present invention relates to microparticles M which can be obtained by means of the process according to the invention and also to an agrochemical formulation comprising microparticles M. In addition, the present invention relates to the use of microparticles M prepared according to the invention as component in colorants, cosmetics, drugs, biocides, plant protection agents, fertilizers, additives for food or animal fodder, or auxiliaries for polymers, paper, textiles, leather, detergents or cleaners. Finally, the invention also relates to a method for combating undesirable plant growth, to a method for combating undesirable insect or acarid infestation on plants and/or for combating phytopathogenic fungi, and to seeds treated with the agrochemical formulation.

A subject matter of the present invention is a process for the preparation of effect compound-comprising microparticles M comprising A) the formation of a crude suspension of microparticles A by means of enzymatic polyester synthesis in an inverse miniemulsion comprising enzyme, effect compounds and polyester monomers; and B) the polymerization of wall monomers from the group consisting of ethylenically unsaturated monomers, polyisocyanates and polyepoxides in the crude suspension of microparticles A. Furthermore, the present invention relates to microparticles M which can be obtained by means of the process according to the invention and also to an agrochemical formulation comprising microparticles M. In addition, the present invention relates to the use of microparticles M prepared according to the invention as component in colorants, cosmetics, drugs, biocides, plant protection agents, fertilizers, additives for food or animal fodder, or auxiliaries for polymers, paper, textiles, leather, detergents or cleaners. Finally the invention also relates to a method for combating undesirable plant growth, to a method for combating undesirable insect or acarid infestation on plants and/or for combating phytopathogenic fungi, and to seed treated with the agrochemical formulation.

Combinations of preferred characteristics with other preferred characteristics are comprised within the present invention.

Microparticles are known in the most varied embodiments and are used for very different purposes according to the impermeability of the capsule wall. For example, they are used for the protection of core materials which are only to be released through selected mechanical destruction of the capsule envelope, for example, of colorant precursors for copy paper or of encapsulated aromatic principles. Capsule envelope materials based on gelatin, polyurethane resin, melamine/formaldehyde resin and polyacrylates are known in such fields of application. Other demands are placed on wall materials for plant or pharmaceutical active compounds as core materials, in which what matters is that the capsule envelope has a porosity which makes it possible to control release and purposeful transportation of the active compounds. In addition to the capsules prepared by chemical processes, mechanical/physical preparation processes are also known here.

Chemical or physical methods are generally known for the preparation of microparticles. With physical methods, dissolved polymers are usually applied to the material to be encapsulated and converted to a solid capsule wall by physical methods, such as spray drying or solvent removal. With chemical methods, the solid capsule wall is formed by chemical reaction, for example, by polymerization of monomers, on the material to be encapsulated. An additional physical stage for the formation of the solid microparticles is not necessary.

Polyester-comprising microparticles and their preparative processes, are generally known. Such microparticles can be prepared starting from polymeric feedstocks for the capsule envelope. Thus, EP 1 421 990 reveals a process for the preparation of microparticles, in which a polyester, which is dispersed in a polyol, is emulsified with an enzyme, as effect compound, which is dispersed in a polyol. U.S. Pat. No. 4,637,905 discloses a process for the preparation of microparticles with a size of 1 to 2000 μm, in which a dispersion of polylactic acid with a protein as an effect compound is prepared, a portion of the solvent is evaporated and, finally, the concentrated dispersion is added to a third solvent for the encapsulating of the effect compound. WO 2002/069922 reveals microparticles with an oxidoreductase-comprising aqueous core and a polyester-comprising envelope. The preparation takes place by emulsifying an aqueous enzyme solution with a polyester dissolved in an organic solvent, followed by introduction of the primary emulsion into an aqueous solvent and subsequently by the removal of the organic solvent. DE 102005007374 reveals nanoparticles of the core-shell type. A polymer which is hydrophobic and biocompatible is defined as shell. The polymer is, for example, polyacrylate, polyepoxide, polyurethane or polyester. An active compound which is enclosed by the polymer of the shell is defined as core. The preparation takes place by radical polymerization, polyaddition, polycondensation or enzymatic or anionic polymerization. Details of the process or examples are not mentioned. PCT/EP2008/054702 reveals a process for the preparation of microcapsules, comprising an effect compound-comprising capsule core and a polymer-comprising capsule envelope, comprising the formation of the capsule envelope by means of enzyme-catalyzed polymerization of monomers present in an inverse miniemulsion. Disadvantages of the known processes are, for example, that the polymers which form the microparticles are prepared separately by polymerization, that the microparticles are not sufficiently stable or that the rate of release of the effect compound cannot be controlled.

It was an object of the present invention to make available an improved process for the preparation of effect compound-comprising microparticles. It was in particular an object of the present invention to make available a process in which polyester-comprising microparticles can be provided with improved stability of the capsule structure. An additional aspect of the object was to prepare the abovementioned microparticles under mild reaction conditions, so that even sensitive effect compounds can be encapsulated. A further aspect was that the later release of the effect compound could be controlled through the preparation process and the monomer composition.

The object was achieved through a process for the preparation of effect compound-comprising microparticles M comprising A) the formation of a crude suspension of microparticles A by means of enzymatic polyester synthesis in an inverse miniemulsion comprising enzyme, effect compound and polyester monomers; and B) the polymerization of wall monomers from the group consisting of ethylenically unsaturated monomers, polyisocyanates and polyepoxides in the crude suspension of microparticles A.

An ensemble of microparticles M is generally prepared by means of the process according to the invention. The process according to the invention generally results in identically or similarly shaped microparticles. Microparticles prepared according to the invention can take any shape. Preferably, they are essentially constructed spherically, for example, perfectly spherically.

Effect compound-comprising microparticles M prepared according to the invention usually have the structure of a capsule or of a matrix particle, preferably of a capsule. Capsules are typically formed from a polymer-comprising capsule envelope and an effect compound-comprising capsule core. Matrix particles are generally formed from a polymer-comprising particle core, in which an effect compound is present in finely distributed form.

According to the invention, a capsule should also be obtained which comprises at least one capsule envelope and at least one capsule core. Thus, a capsule can, for example, exhibit one capsule core and two capsule envelopes. Likewise, a capsule can, for example, exhibit several capsule cores, for example two capsule cores next to one another or one within the other, and one capsule envelope, for example, two capsule envelopes next to one another or one within the other. Preferably, a capsule comprises one capsule envelope and one capsule core. The thickness of the capsule envelope can vary within a wide range. It is generally from 0.1 to 90%, preferably from 0.5 to 20%, of the capsule radius (determinable via light/electron microscopy or light scattering).

The mean diameter of the microparticles M (determinable as Z-mean by means of light scattering of a 1% by weight aqueous dispersion of microparticles, obtainable by diluting the microparticle suspension with water and, if appropriate, separating an organic phase) can vary within a wide range. It is generally more than 0.1 μm, preferably more than 0.6 μm, particularly preferably more than 0.8 μm. The diameter preferably ranges from 0.1 to 2000 μm, preferably from 0.6 to 1000 μm, in particular from 0.8 to 800 μm. A diameter lying in the lower range is preferred if a relatively high mechanical stability is desired for the microparticles. A diameter in the higher range is preferred in order to pack as much capsule content as possible in as little wall material as possible.

The microparticles M usually comprise at least one effect compound. The effect compound is present in this connection in the particle core or in the capsule core generally in solid, dissolved, emulsified or dispersed form. In a preferred embodiment, the capsule core comprises at least one effect compound and at least one inert compound, which is preferably a liquid. All compounds present in the process according to the invention, for example, are suitable as inert compound: dispersants, polar and/or nonpolar liquids, water or enzymes which have a catalytic effect. The capsule core comprises in particular at least one effect compound and at least one polar solvent. The particle core or the capsule core can also comprise incompletely polymerized monomer. According to a preferred embodiment, the capsule core comprises at least the polar liquid, which forms the dispersed phase of the inverse miniemulsion.

Use is made according to the invention, in the process for the preparation of the microparticles M, of enzymes which catalyze the polymerization of the polyester monomers. Use is made, for the description of enzymes, of EC Classes developed by the “Nomenclature Committee of the International Union of Biochemistry and Molecular Biology (NC-IUBMB)”. It is obviously possible to use an individual hydrolase or a mixture of different hydrolases. It is also possible to use the hydrolases in the free and/or immobilized form.

Examples of suitable hydrolases [EC 3.x.x.x] are esterases [EC 3.1.x.x], proteases [EC 3.4.x.x], hydrolases which react with other C—N bonds than peptide bonds [EC 3.5.x.x] or hydrolases which react with acid anhydrides [EC 3.6.x.x]. Use is advantageously made, according to the invention, of in particular carboxylesterases [EC 3.1.1.1], lipases [EC 3.1.1.3] or cutinases [EC 3.1.1.47]. Examples for this are lipases from Achromobacter sp., Aspergillus sp., Candida sp., Candida antarctica, Mucor sp., Penicillium sp., Geotricum sp., Rhizopus sp., Burkholderia sp., Pseudomonas sp., Pseudomonas cepacia, Thermomyces sp., porcine pancreas or wheat germ and carboxylesterases from Bacillus sp., Pseudomonas sp., Burkholderia sp., Mucor sp., Saccharomyces sp., Rhizopus sp., Thermoanaerobium sp., pig liver or horse liver. Use is preferably made of lipase from Pseudomonas cepacia, Burkholderia platarii or Candida antarctica type B in free or immobilized form (for example Novozym® 435 from Novozymes A/S, Denmark).

The total amount of the enzymes used is generally from 0.001 to 40% by weight, frequently from 0.1 to 15% by weight and often from 0.5 to 10% by weight, in each case based on the total amount of polyester monomers. The amount depends on the purity of the enzyme used. Industrial or immobilized enzymes are generally used in higher amounts than purified enzymes. The person skilled in the art will also tell, according to amount of catalyst, how quickly the reaction should come to an end.

Suitable polyester monomers are, for example, hydroxycarboxylic acid compounds, dialcohol compounds or diacid compounds, in particular hydroxycarboxylic acid compounds. A combination of the preceding monomers is likewise possible, the combination of dialcohol compounds and diacid compounds being preferred.

In a preferred embodiment, the polyester monomers are combined with an initiator monomer which is a hydracid compound, such as hydroxy- or amino-functional compounds or water. A suitable initiator monomer is a hydroxycarboxylic acid compound, dialcohol compound or diacid compound. The initiator monomer is preferably a dialcohol compound as described below, in particular ethylene glycol, 1,4-butanediol, glycerol, sorbitol, monosaccharide, disaccharide, polysaccharide or hydroxy-functional, dendritic polyesters based on 2,2-dimethylolpropionic acid (Boltorn® grades, commercially available from Perstorp).

Use may be made, as hydroxycarboxylic acid compounds, of the free hydroxycarboxylic acids with at least one free alcohol group and at least one free carboxylic acid group, their C₁-C₅ alkyl esters and/or their lactones. Mention may be made, by way of example, of glycolic acid, D-, L-, or D,L-lactic acid, 6-hydroxyhexanoic acid (6-hydroxycaproic acid), 3-hydroxybutyric acid, 3-hydroxyvaleric acid, 3-hydroxycaproic acid, or their cyclic derivatives, such as glycolide (1,4-dioxane-2,5-dione), D-, L-, or D,L-dilactide (3,6-dimethyl-1,4-dioxane-2,5-dione), ε-caprolactone, β-butyrolactone, γ-butyrolactone, ω-dodecanolide (oxacyclotridecane-2-one), ω-undecanolide (oxacyclododecane-2-one) or ω-pentadecanolide (oxacyclohexadecane-2-one). Bis- or trislactones, which comprise two or three lactone groups, are also suitable as lactones. By way of example, (2,2′-bis(ε-caprolacton-4-yl)propane can be used. Bislactones can, for example, be synthesized according to Palmgren et al., Journal of Polymer Science A, 1997, 35, 1635-1649. The esters of carbonic acid (carbonates), in particular linear and cyclic aliphatic carbonates, preferably C₁- to C₈-alkyl esters of carbonic acid, in particular trimethylene carbonate are likewise suitable. Carbonates which do not react with the respective enzyme, for example propylene carbonate, are not suitable as monomer. Use may also be made, as hydroxycarboxylic acid compounds, of the thiocarboxylic acid and its esters and thiolactones analogous to the abovementioned hydrocarboxylic acid compounds. Mixtures of different hydroxycarboxylic acid compounds can obviously also be used. Preferred hydroxycarboxylic acid compounds are lactones, in particular C₂- to C₁₈-alkylenelactones, very particularly preferably ε-caprolactone.

Use may be made in principle, as dicarboxylic acid compounds, of all C₂-C₄₀ aliphatic, C₃-C₂₀ cycloaliphatic, aromatic or heteroaromatic compounds exhibiting at least two carboxylic acid groups (carboxyl groups; —COOH) or derivatives thereof. C₁-C₁₀-Alkyl, preferably methyl, ethyl, n-propyl or isopropyl, mono- or diesters of the abovementioned dicarboxylic acids, and also the corresponding dicarboxylic acid anhydrides, are in particular of use as derivatives. Examples of dicarboxylic acid compounds are ethanedioic acid (oxalic acid), propanedioic acid (malonic acid), butanedioic acid (succinic acid), pentanedioic acid (glutaric acid), hexanedioic acid (adipic acid), heptanedioic acid (pimelic acid), octanedioic acid (suberic acid), nonanedioic acid (azelaic acid), decanedioic acid (sebacic acid), undecanedioic acid, dodecanedioic acid, tridecanedioic acid (brassylic acid), C₃₂ dimer fatty acid, benzene-1,2-dicarboxylic acid (phthalic acid), benzene-1,3-dicarboxylic acid (isophthalic acid) or benzene-1,4-dicarboxylic acid (terephthalic acid), their methyl esters, for example dimethyl ethanedioate, dimethyl propanedioate, dimethyl butanedioate, dimethyl pentanedioate, dimethyl hexanedioate, dimethyl heptanedioate, dimethyl octanedioate, dimethyl nonanedioate, dimethyl decanedioate, dimethyl undecanedioate, dimethyl dodecanedioate, dimethyl tridecanedioate, C₃₂ dimer fatty acid dimethyl ester, dimethyl phthalate, dimethyl isophthalate or dimethyl terephthalate, and their anhydrides, for example butanedicarboxylic anhydride, pentanedicarboxylic anhydride or phthalic anhydride. Use can obviously also be made of mixtures of the abovementioned dicarboxylic acid compounds. Oligoesters and polyesters with at least two free carboxyl groups, in particular carboxy-terminated oligo- and polyesters, can likewise be used as dicarboxylic acid component. Use can likewise also be made of the esters of polycarboxylic acids, such as, for example, citric acid and butanetetracarboxylic acid. Use is preferably made of the free dicarboxylic acids, in particular C₄ to C₃₆ aliphatic dicarboxylic acids, in particular butanedioic acid, hexanedioic acid, decanedioic acid or dodecanedioic acid or their corresponding dimethyl and diethyl esters.

Use may be made, as diol compounds, of branched or linear alkanes with 2 to 18 carbon atoms, preferably 4 to 14 carbon atoms, cycloalkanes with 5 to 20 carbon atoms or aromatic compounds comprising at least two alcohol groups. Examples of suitable alkane diols are ethylene glycol, 1,2-propanediol, 1,3-propanediol, 1,2-butanediol, 1,4-butanediol, 1,5-pentanediol, 1,6-hexanediol, 1,7-heptanediol, 1,8-octanediol, 1,9-nonanediol, 1,10-decanediol, 1,11-undecanediol, 1,12-dodecanediol, 1,13-tridecanediol, 2,4-dimethyl-2-ethyl-1,3-hexanediol, 2,2-dimethyl-1,3-propanediol (neopentyl glycol), 2-ethyl-2-butyl-1,3-propanediol, 2-ethyl-2-isobutyl-1,3-propanediol or 2,2,4-trimethyl-1,6-hexanediol. Ethylene glycol, 1,3-propanediol, 1,4-butanediol, 2,2-dimethyl-1,3-propanediol, 1,6-hexanediol or 1,12-dodecanediol are suitable in particular. Examples of cycloalkanediols are 1,2-cyclopentanediol, 1,3-cyclopentanediol, 1,2-cyclohexanediol, 1,3-cyclohexanediol, 1,4-cyclohexanediol, 1,2-cyclohexanedimethanol (1,2-dimethylolcyclohexane), 1,3-cyclohexanedimethanol (1,3-dimethylolcyclohexane), 1,4-cyclohexanedimethanol (1,4-dimethylolcyclohexane) or 2,2,4,4-tetramethyl-1,3-cyclobutanediol. Examples of suitable aromatic diols are 1,4-dihydroxybenzene, 1,3-dihydroxybenzene, 1,2-dihydroxybenzene, bisphenol A (2,2-bis(4-hydroxyphenyl)propane), 1,3-dihydroxynaphthalene, 1,5-dihydroxynaphthalene or 1,7-dihydroxynaphthalene. However, use may also be made, as diol compounds, of polyetherdiols, for example diethylene glycol, triethylene glycol, polyethylene glycol (with more than 4 ethylene oxide units), propylene glycol, dipropylene glycol, tripropylene glycol, polypropylene glycol (with more than 4 propylene oxide units) and polytetrahydrofuran (poly-THF), in particular diethylene glycol, triethylene glycol and polyethylene glycol (with more than 4 ethylene oxide units). Compounds having a number-average molecular weight (Mn) generally ranging from 200 to 10 000 g/mol, preferably from 600 to 5000 g/mol, are of use as poly-THF, polyethylene glycol or polypropylene glycol. Oligoesters and polyesters with at least two free alcohol groups, preferably dihydroxy-terminated oligo- and polyesters, are likewise suitable. Also suitable are dendrimers, exhibiting at least two free primary or secondary alcohol groups. Also suitable are polycarbonates, exhibiting at least two free primary or secondary alcohol groups. Additional examples of suitable diol compounds with more than two alcohol groups are glycerol, sorbitol, trimethylolpropane, pentaerythritol, monosaccharides, such as fructose, glucose or mannose, disaccharides such as sucrose, oligosaccharides and their substitution products, or cellulose derivatives such as acetates. Use may also be made, as diol compounds, of a dithiol analogous to the abovementioned diol compounds. Mixtures of the abovementioned diol compounds or dithiols can obviously also be used. Preferred diols are aliphatic alkanediols and polyetherdiols, particularly preferably linear and branched aliphatic alkanediols with 2 to 18 carbon atoms, in particular ethylene glycol, 1,4-butanediol, 1,6-hexanediol, sorbitol and neopentyl glycol.

Linear, branched or crosslinked polyesters can be produced from the monomers described above, depending on whether difunctional monomers or higher functionality monomers are used.

The polyester monomers are generally present in the reaction mixture in stage A) at from 0.1 to 20% by weight, preferably at from 0.5 to 10% by weight, in particular at from 1 to 5% by weight, based on the total charge. In a preferred embodiment, at least one lactone is present at from 0.1 to 20% by weight, preferably at from 0.5 to 10% by weight, in particular at from 1 to 5% by weight, based on the total charge in stage A).

Dispersants can be used according to the process according to the invention. These can in principle be protective colloids, emulsifiers or their mixtures. In this connection it is obvious that the emulsifiers and/or protective colloids are chosen so that they are in particular compatible with the enzymes used and do not deactivate them.

The polymerization can be carried out in the presence of protective colloids, if appropriate also in addition to emulsifiers. They generally have average molar masses Mw of greater than 500 g/mol, preferably of more than 1000 g/mol. Examples of protective colloids are polyvinyl alcohols, cellulose derivatives, such as carboxymethylcellulose, polyvinylpyrrolidone, polyethylene glycols, graft polymers of vinyl acetate and/or vinyl propionate on polyethylene glycols, polyethylene glycols closed at one or both ends with alkyl, carboxyl or amino groups, poly(diallyldimethylammonium chloride)s and/or polysaccharides, such as in particular water-soluble starches or starch derivatives.

Use is frequently made exclusively of emulsifiers as dispersants. Use is generally made of emulsifiers having a relative molecular weight, unlike the protective colloids, of usually less than 1000 g/mol. They can be anionic, cationic or nonionic in nature. In the case of the use of mixtures of surface-active substances, the individual components must obviously be compatible with one another, which, in case of doubt, can be verified from a few preliminary tests. In general, anionic emulsifiers are compatible with one another and with nonionic emulsifiers. The same also applies for cationic emulsifiers, while anionic and cationic emulsifiers are generally not compatible with one another.

The polymerization can, if appropriate, also be carried out in the presence of finely divided water-insoluble inorganic emulsifiers (“Pickering emulsifiers”), for example barium sulfate.

Conventional nonionic emulsifiers are, e.g. ethoxylated mono-, di- and trialkylphenols (degree of ethoxylation from 3 to 50, alkyl radical: C₄ to C₁₂) and ethoxylated fatty alcohols (degree of ethoxylation from 3 to 80; alkyl radical: C₈ to C₃₆). Examples of these are the Lutensol® A brands (C₁₂ to C₁₄ fatty alcohol ethoxylates, degree of ethoxylation from 3 to 8), Lutensol® AO brands (C₁₃ to C₁₅ oxo alcohol ethoxylates, degree of ethoxylation from 3 to 30), Lutensol® AT brands (C₁₆ to C₁₈ fatty alcohol ethoxylates, degree of ethoxylation from 11 to 80), Lutensol® ON brands (C₁₀ oxo alcohol ethoxylates, degree of ethoxylation from 3 to 11) and Lutensol® TO brands (C₁₃ oxo alcohol ethoxylates, degree of ethoxylation from 3 to 20) from BASF SE.

Conventional anionic emulsifiers are, e.g., alkali metal and ammonium salts of alkyl sulfates (alkyl radical: C₈ to C₁₂), of sulfuric acid monoesters of ethoxylated alkanols (degree of ethoxylation from 4 to 30, alkyl radical: C₁₂ to C₁₈) and ethoxylated alkylphenols (degree of ethoxylation from 3 to 50, alkyl radical: C₄ to C₁₂), of alkylsulfonic acids (alkyl radical: C₁₂ to C₁₈) and of alkylarylsulfonic acids (alkyl radical: C₉ to C₁₈). Furthermore, compounds of the general formula (I)

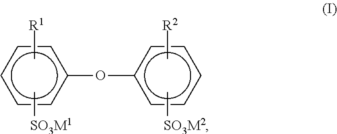

in which R¹ and R² represent hydrogen atoms or C₄-C₂₄-alkyl and are not simultaneously hydrogen atoms, and M¹ and M² can be alkali metal ions and/or ammonium ions, have proven to be additional anionic emulsifiers. In the general formula (I), R¹ and R² preferably represent linear or branched alkyl radicals with from 6 to 18 carbon atoms, in particular with 6, 12 and 16 carbon atoms or hydrogen, R¹ and R² not both simultaneously being hydrogen atoms. M¹ and M² are preferably sodium, potassium or ammonium, sodium being particularly preferred. Compounds (I) in which M¹ and M² are sodium, R¹ is a branched alkyl radical with 12 carbon atoms and R² is a hydrogen atom or R¹ are particularly advantageous. Use is frequently made of industrial mixtures exhibiting a proportion of the monoalkylated product of from 50 to 90% by weight, such as, for example Dowfax® 2A1 (brand of Dow Chemical Company).

Suitable cationic emulsifiers are generally cationic salts exhibiting a C₆-C₁₈-alkyl, C₆-C₁₈-alkylaryl or heterocyclic radical, for example primary, secondary, tertiary or quaternary ammonium salts, alkanolammonium salts, pyridinium salts, imidazolinium salts, oxazolinium salts, morpholinium salts, thiazolinium salts and salts of amine oxides, quinolinium salts, isoquinolinium salts, tropylium salts, sulfonium salts and phosphonium salts. Mention may be made, by way of example, of dodecylammonium acetate or the corresponding sulfate, the sulfates or acetates of the various 2-(N,N,N-trimethylammonio)ethylparaffinic acid esters, N-cetylpyridinium sulfate, N-laurylpyridinium sulfate, N-cetyl-N,N,N-trimethylammonium sulfate, N-dodecyl-N,N,N-trimethylammonium sulfate, N-octyl-N,N,N-trimethylammonium sulfate, N,N-distearyl-N,N-dimethylammonium sulfate, the Gemini surfactant N,N′-dilauryl-N,N,N′,N′-tetramethylethylenediamine disulfate, ethoxylated (tallow fatty alkyl)-N-methylammonium sulfate and ethoxylated oleylamine (for example Uniperol® AC from BASF Aktiengesellschaft, approximately 12 ethylene oxide units). It is essential for the anionic countergroups to have the lowest possible nucleophilicity, such as, for example, perchlorate, sulfate, phosphate, nitrate and carboxylates, such as acetate, trifluoroacetate, trichloroacetate, propionate, oxalate, citrate or benzoate, and also conjugated anions of organosulfonic acids, such as for example, methylsulfonate, trifluoromethylsulfonate and paraffintoluenesulfonate, furthermore tetrafluoroborate, tetraphenylborate, tetrakis(pentafluorophenyl)borate, tetrakis[bis(3,5-trifluoromethyl)phenyl]borate, hexafluorophosphate, hexafluoroarsenate or hexafluoroantimonate.

Preferred emulsifiers are nonionic emulsifiers, in particular ethoxylated alcohols and sorbitan esters, particularly preferably ethoxylated fatty alcohols and sorbitan fatty acid esters. Very particularly preferred mixtures comprise ethoxylated alcohols and sorbitan esters. In a preferred embodiment, the mixtures comprise ethoxylated alcohols and sorbitan esters.

In an additional preferred embodiment, a polymer based on the final reaction product of polyisobutylene and maleic anhydride (PIBSA) and di(alkyl)ethanolamine is suitable. In an additional preferred embodiment, block copolymers are suitable, such as are described in Macromolecules 38 (16), 6882-6887, block copolymers based on isoprene and methyl methacrylate such as are described in WO 2008/009424, or poly((ethylene-co-butylene)-block-ethylene oxide).

The emulsifiers preferably used as dispersants are advantageously used in a total amount of from 0.005 to 20% by weight, preferably from 0.01 to 15% by weight, in particular from 0.1 to 10% by weight, in each case based on the total charge in stage A). The total amount of the protective colloids used as dispersants in addition to or in place of the emulsifiers is often from 0.1 to 10% by weight and frequently from 0.2 to 7% by weight, in each case based on the total charge in stage A).

The inverse miniemulsion, in accordance with the invention in which the polyester monomers are for the most part present comprises a continuous nonpolar phase and a discontinuous polar phase. The polar phase comprises a polar liquid and the nonpolar phase comprises a nonpolar liquid. The effect compound is essentially present in the discontinuous phase in solid, dissolved, emulsified or dispersed form. The polyester monomers, dispersants or enzymes can be present both distributed only in one of the two phases and in both phases, or at the interface of the two phases. In a preferred embodiment, the polyester monomer is present in the polar phase at least 70% by weight, preferably at least 80% by weight and in particular at least 90% by weight, in each case based on the total amount of the polyester monomers in stage A). In an additional preferred embodiment, the polar liquid is composed of at least one polyester monomer and at least one effect compound.

The mean size of the droplets of the discontinuous phase of the inverse miniemulsion according to the invention can preferably be determined according to the principle of quasielastic dynamic light scattering on a 1% by weight miniemulsion, obtainable by diluting the inverse miniemulsion with the corresponding continuous phase and, if appropriate, separating an organic phase (what is known as the Z-mean droplet diameter d_{z} of the unimodal analysis of the autocorrelation function). Additional methods of determination are light or electron microscopy, and also field flow fractionation. The values for d_{z} thus determined for the inverse miniemulsions are, according to the invention, normally less than 10 000 nm, frequently less than 1000 nm, and generally less than 500 nm. The d_{z} range, according to the invention, is conveniently from 2000 nm to 1000 nm. Normally, d_{z} of the inverse miniemulsion to be used according to the invention is greater than 40 nm.

Suitable polar liquids are those having a solubility in the continuous nonpolar phase under reaction conditions of less than 40% by weight, preferably less than 10% by weight and in particular less than 1% by weight (in each case based on the total amount of the continuous phase), so that a separate discontinuous polar phase is present. In a preferred embodiment, the polar liquid dissolves the polyester monomer at 20° C. to at most 10% by weight, preferably to at most 3% by weight and in particular to at most 0.5% by weight, in each case based on the total weight of the polyester monomer.

Suitable polar liquids are, for example, monools, such as C₃-C₆-alkanols, in particular tert-butanol and tert-amyl alcohol, pyridine, poly-C₁-C₄-alkylene glycol di-C₁-C₄-alkyl ethers, in particular polyethylene glycol di-C₁-C₄-alkyl ethers, such as e.g., dimethoxyethane, diethylene glycol dimethyl ether or polyethylene glycol dimethyl ether 500, C₂-C₄-alkylene carbonates, in particular propylene carbonate, C₃-C₆-alkyl acetates, in particular tert-butyl acetate, acetone, 1,4-dioxane, 1,3-dioxolane, tetrahydrofuran, dimethoxymethane, dimethoxyethane, aqueous buffers or water. It is obviously also possible to use mixtures of the abovementioned solvents. The abovementioned polyester monomers or their mixtures are also suitable polar liquids.

The polar liquid can, for example, also comprise the effect compound used or it can consist of it. Propylene carbonate and mixtures comprising propylene carbonate are a preferred polar liquid. In an additional preferred embodiment, the polyester monomer is the polar liquid.

If a lactone is used as polyester monomer, the polar liquid comprises less than 5% by weight, preferably less than 1% by weight and in particular less than 0.1% by weight of water. If the polar liquid comprises water, it is advantageous for the aqueous reaction medium to exhibit, at ambient temperature (20 to 25° C.), a pH from 2 to 11, frequently from 3 to 9 and often from 6 to 8. In particular, in the aqueous reaction medium, the pH is adjusted to a value at which the enzyme exhibits a high catalytic activity and a high service life. The appropriate measures for adjusting the pH, i.e. addition of appropriate amounts of acid, for example, sulfuric acid, bases, for example, aqueous solutions of alkali metal hydroxides, in particular sodium hydroxide or potassium hydroxide, or buffering agents, for example, potassium dihydrogenphosphate/disodium hydrogenphosphate, acetic acid/sodium acetate, ammonium hydroxide/ammonium chloride, potassium dihydrogenphosphate/sodium hydroxide, borax/hydrochloric acid, borax/sodium hydroxide or tris(hydroxymethyl)aminomethane/hydrochloric acid, are familiar to a person skilled in the art.

In order to further increase the polarity of the polar phase, it can additionally comprise “hydrophilic agents”. Suitable hydrophilic agents are, for example, organic or inorganic salts or highly polar neutral compounds. Examples of inorganic salts are sodium nitrite, sodium chloride, potassium chloride, lithium chloride or rubidium chloride. Examples of organic salts are trialkylammonium salts, ionic liquids, such as ethylmethylimidazolium salts, or oligomers with stoichiometric proportions of anionic and cationic groups in the main or side chain. Preference is given to hydrophilic agents which do not reduce the catalytic activity of the enzymes.

Suitable nonpolar liquids are those having a solubility in the discontinuous polar phase under reaction conditions, of less than 10% by weight, preferably of less than 1% by weight and in particular of less than 0.1% by weight (in each case based on the total amount of the continuous phase), so that a separate continuous polar phase is present.

Suitable nonpolar liquids are, for example, aliphatic or aromatic liquid hydrocarbons with from 5 to 30 carbon atoms, for example, n-pentane and isomers, cyclopentane, n-hexane and isomers, cyclohexane, n-heptane and isomers, n-octane and isomers, n-nonane and isomers, n-decane and isomers, n-dodecane and isomers, n-tetradecane and isomers, n-hexadecane and isomers, n-octadecane and isomers, benzene, toluene, ethylbenzene, cumene, o-, m- or p-xylene or mesitylene. Hydrocarbon mixtures in the boiling point range from 30 to 250° C., such as partially hydrogenated mineral oil distillates (e.g. Isopar® brands, Exxon Mobil), are also suitable. Olefins, for example, polyisobutylenes or C₆-C₃₀-α-olefins, are also suitable. Hydroxy compounds, such as saturated and unsaturated fatty alcohols with from 10 to 28 carbon atoms, for example, n-dodecanol, n-tetradecanol, n-hexadecanol and their isomers or cetyl alcohol, or esters, such as, for example, fatty acid esters with from 10 to 28 carbon atoms in the acid part and from 1 to 10 carbon atoms in the alcohol part or esters of carboxylic acids and fatty alcohols with from 1 to 10 carbon atoms in the carboxylic acid part and from 10 to 28 carbon atoms in the alcohol part, can likewise be used. Additional suitable nonpolar liquids are liquid paraffin (linear hydrocarbon mixtures), silicone oil (polysiloxane), perfluorinated hydrocarbons, fluorosilicone oil, perfluorinated polyethers, fluorosilane or siloxane, such as dimethylsiloxane. Aliphatic and aromatic liquid hydrocarbons with from 5 to 30 carbon atoms, in particular partially hydrogenated mineral oil distillates, are preferred nonpolar liquids. In an additional embodiment, nonpolar liquids are liquid paraffin. It is obviously also possible to use mixtures of the abovementioned solvents.

The total amount of polar and nonpolar liquids is chosen in such a way that the total charge in stage A) reaches 100% by weight. It is generally from 10 to 90% by weight, preferably from 40 to 70% by weight, based on the total charge.

The ratio by weight of polar to nonpolar liquid is in this connection chosen so that a discontinuous phase is produced, essentially comprising the polar liquid. In a preferred embodiment, use is made of from 20 to 80% by weight, preferably from 40 to 70% by weight, of nonpolar liquid, in each case based on the total charge. In an additional preferred embodiment, use is made of from 20 to 80% by weight, preferably from 30 to 60% by weight of polar liquid, in each case based on the total charge. In an additional preferred embodiment, use is made of from 20 to 80% by weight, preferably from 35 to 55% by weight, of hydrocarbon mixtures and from 20 to 70% by weight, preferably from 30 to 60% by weight, of propylene carbonate, in each case based on the total charge. Care is to be taken in this connection that the miniemulsions do not suffer a phase inversion, i.e. that the hydrophobic continuous phase does not become the disperse phase.

The term “effect compounds” should be understood as meaning, in the context of the invention, compounds which cause effects desired by the user in the industrial application of the product according to the invention. Effect compounds are, for example, colorants, cosmetics, drugs, biocides, plant protection agents, agrochemical adjuvants, fertilizers, additives for food or animal fodder, or auxiliaries for polymers, paper, textiles, leather, detergents or cleaners. The person skilled in the art can, depending upon the field of application desired, choose the appropriate effect compound on the basis of his general specialized knowledge.

Examples of colorants are dyes, printing inks, pigments, UV absorbers, optical brighteners or IR dyes. While organic dyes have an absorption maximum in the wavelength range from 400 to 850 nm, optical brighteners have one or more absorption maxima in the range from 250 to 400 nm. Optical brighteners are known to emit, on irradiating with UV light, a fluorescence in the visible region. Examples of optical brighteners are compounds from the categories of the bisstyrylbenzenes, stilbenes, benzoxazols, coumarins, pyrenes and naphthalenes. Marking compounds for liquids, for example, mineral oil markers, are also suitable. The term “UV absorbers” is understood as generally meaning compounds which absorb UV radiation, which nonradiatively deactivate the absorbed radiation. Such compounds are used, for example, in sunscreens and for the stabilizing of organic polymers.

Cosmetics are additional suitable effect compounds. Cosmetics are compounds or compositions of compounds which are exclusively or predominantly intended to be applied externally on the human body or in the human oral cavity for cleaning, caring, protecting, maintaining a good condition, scenting or changing the appearance or are to be applied thereto to influence the body odor. Insect repellants, such as, Icaridin® or N,N-diethyl-meta-toluamide (DEET®), are also suitable, for example.

In addition, all drugs can be used as effect compounds.

The following may be included as biocides: biocides comprising heavy metals, such as (N-cyclohexyldiazeniumdioxy)tributyltin or bis(N-cyclohexyldiazeniumdioxy)copper (CuHDO); metal soaps, such as tin, copper, or zinc naphthenate, octoate, 2-ethylhexanoate, oleate, phosphate or benzoate; metal salts, such as basic copper carbonate, sodium dichromate, potassium dichromate, potassium chromate, copper sulfate, copper chloride, copper borate, zinc fluorosilicate, copper fluorosilicate or copper salt of 2-pyridinethiol 1-oxide; oxides, such as tributyltin oxide, Cu₂O, CuO or ZnO; or zeolites comprising Ag, Zn or Cu, alone or enclosed in polymeric active compounds. Suitable biocides are preferably algaecides, such as diuron, dichlorophen, endothal, fentin acetate or quinoclamine, molluscicides such as fentin acetate, metaldehyde, methiocarb, niclosamide, thiodicarb and trimethacarb, fungicides, such as dithianon, bronopol, dichlofluanid, tolylfluanid, iodopropargyl butylcarbamate, fluorofolpet and azoles, such as tebuconazole or conventional antifouling active compounds, such as 2-(N,N-dimethylthiocarbamoylthio)-5-nitrothiazole, tetrabutyldistannoxane, 2-(tert-butyl)amino-4-cyclopropylamino-6-methylthio-1,3,5-triazine, 4,5-dichloro-2-(n-octyl)-4-isothiazolin-3-one, 2,4,5,6-tetrachloro-isophthalodinitrile, tetramethylthiuram disulfide, 2,4,6-trichlorophenylmaleimide, 2,3,5,6-tetrachloro-4-(methylsulfonyl)pyridine, diiodomethyl para-tolyl sulfone, thiabendazole, pyridinium tetraphenylborate, or sodium salt of 2-pyridinethiol 1-oxide. An additional example is sodium chlorite (NaClO2) or 2,4-dichlorobenzyl alcohol (DCBA). Preferred biocides are bis(N-cyclohexyldiazeniumdioxy)copper, dithianon, bronopol, sodium chlorite (NaClO₂) or 2,4-dichlorobenzyl alcohol.

The microcapsules according to the invention comprising biocides can be used wherever what matters is that surfaces should be as free as possible from bacteria, algae and fungi, i.e. microbiocidal surfaces, or that surfaces should have nonstick properties. They can be used in the following fields
marine: ship hulls, docks, buoys, drilling platforms, ballast water tanks;
house: roofing, basements, walls, facades, greenhouses, protection against the sun, garden fences, wood preservation;
sanitary: comfort stations, bathrooms, shower curtains, toiletries, swimming pools, saunas, joints, jointing compounds;
foodstuffs: machines, kitchens, kitchenware, sponges, toys, food packaging, milk processing, drinking water systems, cosmetics;
machine parts: air conditioning systems, ion exchangers, process water, solar installations, heat exchangers, bioreactors, membranes, cooling water treatments;
medical engineering: contact lenses, diapers, membranes, implants;
commodities: automobile seats, clothing (stockings, sportswear), hospital equipment, door handles, telephone handsets, public transport, animal cages, cash registers, carpeting, wallpaper.

Plant protection agents and fertilizers can also be used as effect compounds. Acaricides, algaecides, aphicides, bactericides, fungicides, herbicides, insecticides, molluscicides, nematicides, germination inhibitors, safeners or growth regulators are suitable plant protection agents. Fungicides are compounds which destroy fungi and their spores or inhibit their growth. Insecticides are compounds which, in their effect, are directed in particular against insects and their developmental forms. The term “herbicides” is understood to mean compounds which are active against generally all wild and crop plants which are undesirable in their respective locations (harmful plants).

Examples of fertilizers are inorganic single or multi-nucleant fertilizers, organic and organic/inorganic fertilizers or fertilizers with trace elements.

In a preferred embodiment, the effect compounds are plant protection agents or mixtures of plant protection agents. In an additional preferred embodiment, the plant protection agents are preferably herbicides, growth regulators, insecticides or fungicides. It is generally known against which undesirable plants, insects or fungi a plant protection agent can advantageously be used. The following list of plant protection agents demonstrates possible active compounds but is not to be limited to these.

Use may be made, as fungicide, for example of:

A) Strobilurins
azoxystrobin, dimoxystrobin, enestroburin, fluoxastrobin, kresoxim-methyl, metominostrobin, orysastrobin, picoxystrobin, pyraclostrobin, pyribencarb, trifloxystrobin, 2-(2-(6-(3-chloro-2-methylphenoxy)-5-fluoropyrimidin-4-yloxy)phenyl)-2-methoxyimino-N-methylacetamide, methyl 2-(ortho-((2,5-dimethylphenyl)oxymethylene)phenyl)-3-methoxyacrylate, methyl 3-methoxy-2-(2-(N-(4-methoxyphenyl)cyclopropanecarboximidoylsulfanylmethyl)phenyl)acrylate, 2-(2-(3-(2,6-dichlorophenyl)-1-methylallylideneaminooxymethyl)phenyl)-2-methoxyimino-N-methylacetamide;

B) Carboxamides:
carboxanilides: benalaxyl, benalaxyl-M, benodanil, bixafen, boscalid, carboxin, fenfuram, fenhexamide, flutolanil, furametpyr, isopyrazam, isotianil, kiralaxyl, mepronil, metalaxyl, metalaxyl-M (mefenoxam), ofurace, oxadixyl, oxycarboxin, penthiopyrad, tecloftalam, thifluzamide, tiadinil, 2-amino-4-methylthiazole-5-carboxanilide, 2-chloro-N-(1,1,3-trimethylindan-4-yl)nicotinamide, N-(2′,4′-difluorobiphenyl-2-yl)-3-difluoromethyl-1-methyl-1H-pyrazole-4-carboxamide, N-(2′,4′-dichlorobiphenyl-2-yl)-3-difluoromethyl-1-methyl-1H-pyrazole-4-carboxamide, N-(2′,5′-difluorobiphenyl-2-yl)-3-difluoromethyl-1-methyl-1H-pyrazole-4-carboxamide, N-(2′,5′-dichlorobiphenyl-2-yl)-3-difluoromethyl-1-methyl-1H-pyrazole-4-carboxamide, N-(3′,5′-difluorobiphenyl-2-yl)-3-difluoromethyl-1-methyl-1H-pyrazole-4-carboxamide, N-(3′,5′-dichlorobiphenyl-2-yl)-3-difluoromethyl-1-methyl-1H-pyrazole-4-carboxamide, N-(3′-fluorobiphenyl-2-yl)-3-difluoromethyl-1-methyl-1H-pyrazole-4-carboxamide, N-(3′-chlorobiphenyl-2-yl)-3-difluoromethyl-1-methyl-1H-pyrazole-4-carboxamide, N-(2′-fluorobiphenyl-2-yl)-3-difluoromethyl-1-methyl-1H-pyrazole-4-carboxamide, N-(2′-chlorobiphenyl-2-yl)-3-difluoromethyl-1-methyl-1H-pyrazole-4-carboxamide, N-(3′,4′,5′-trifluorobiphenyl-2-yl)-3-difluoromethyl-1-methyl-1H-pyrazole-4-carboxamide, N-(2′,4′,5′-trifluorobiphenyl-2-yl)-3-difluoromethyl-1-methyl-1H-pyrazole-4-carboxamide, N-[2-(1,1,2,3,3,3-hexafluoropropoxy)phenyl]-3-difluoromethyl-1-methyl-1H-pyrazole-4-carboxamide, N-[2-(1,1,2,2-tetrafluoroethoxy)phenyl]-3-difluoromethyl-1-methyl-1H-pyrazole-4-carboxamide, N-(4′-trifluoromethylthiobiphenyl-2-yl)-3-difluoromethyl-1-methyl-1H-pyrazole-4-carboxamide, N-(2-(1,3-dimethylbutyl)phenyl)-1,3-dimethyl-5-fluoro-1H-pyrazole-4-carboxamide, N-(2-(1,3,3-trimethylbutyl)phenyl)-1,3-dimethyl-5-fluoro-1H-pyrazole-4-carboxamide, N-(4′-chloro-3′,5′-difluorobiphenyl-2-yl)-3-difluoromethyl-1-methyl-1H-pyrazole-4-carboxamide, N-(4′-chloro-3′,5′-difluorobiphenyl-2-yl)-3-trifluoromethyl-1-methyl-1H-pyrazole-4-carboxamide, N-(3′,4′-dichloro-5′-fluorobiphenyl-2-yl)-3-trifluoromethyl-1-methyl-1H-pyrazole-4-carboxamide, N-(3′,5′-difluoro-4′-methylbiphenyl-2-yl)-3-difluoromethyl-1-methyl-1H-pyrazole-4-carboxamide, N-(3′,5′-difluoro-4′-methylbiphenyl-2-yl)-3-trifluoromethyl-1-methyl-1H-pyrazole-4-carboxamide, N-[2-(bicyclopropyl-2-yl)phenyl]-3-difluoromethyl-1-methyl-1H-pyrazole-4-carboxamide, N-[2-(cis-bicyclopropyl-2-yl)phenyl]-3-difluoromethyl-1-methyl-1H-pyrazole-4-carboxamide, N-[2-(trans-bicyclopropyl-2-yl)phenyl]-3-difluoromethyl-1-methyl-1H-pyrazole-4-carboxamide, N-[1,2,3,4-tetrahydro-9-(1-methylethyl)-1,4-methanonaphth-5-yl]-3-difluoromethyl-1-methyl-1H-pyrazole-4-carboxamide;
carboxylic acid morpholides: dimethomorph, flumorph;
benzamides: flumetover, fluopicolide, fluopyram, zoxamide, N-(3-ethyl-3,5,5-trimethylcyclohexyl)-3-formylamino-2-hydroxybenzamide;
other carboxamides: carpropamid, diclocymet, mandipropamid, oxytetracycline, silthiofam, N-(6-methoxypyridin-3-yl)cyclopropanecarboxamide;

C) Azoles:
triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazol, penconazole, propiconazole, prothioconazole, simeconazole, tebuconazole, tetraconazole, triadimefon, triadimenol, triticonazole, uniconazole, 1-(4-chlorophenyl)-2-(1,2,4-triazol-1-yl)cycloheptanol;
imidazoles: cyazofamid, imazalil, imazalil sulfate, pefurazoate, prochloraz, triflumizole;
benzimidazoles: benomyl, carbendazim, fuberidazole, thiabendazole;
others: ethaboxam, etridiazole, hymexazol, 2-(4-chlorophenyl)-N-[4-(3,4-dimethoxyphenyl)isoxazol-5-yl]-2-(prop-2-ynyloxy)acetamide;

D) Nitrogen-comprising heterocyclyl compounds
pyridines: fluazinam, pyrifenox, 3-[5-(4-chlorophenyl)-2,3-dimethylisoxazolidin-3-yl]pyridine, 3-[5-(4-methylphenyl)-2,3-dimethylisoxazolidin-3-yl]pyridine, 2,3,5,6-tetrachloro-4-(methanesulfonyl)pyridine, 3,4,5-trichloropyridine-2,6-dicarbonitrile, N-(1-(5-bromo-3-chloropyridin-2-yl)ethyl)-2,4-dichloronicotinamide, N-((5-bromo-3-chloropyridin-2-yl)methyl)-2,4-dichloronicotinamide;
pyrimidines: bupirimate, cyprodinil, diflumetorim, fenarimol, ferimzone, mepanipyrim, nitrapyrin, nuarimol, pyrimethanil;
piperazines: triforine;
pyrroles: fludioxonil, fenpiclonil;
morpholines: aldimorph, dodemorph, dodemorph acetate, fenpropimorph, tridemorph;
piperidines: fenpropidine;
dicarboximides: fluorimid, iprodione, procymidone, vinclozolin;
non-aromatic 5-membered heterocycles: famoxadone, fenamidone, flutianil, octhilinone, probenazole, 5-amino-2-isopropyl-3-oxo-4-(ortho-tolyl)-2,3-dihydropyrazole-1-thiocarboxylic acid S-allyl ester;
others: acibenzolar-S-methyl, amisulbrom, anilazine, blasticidin-S, captafol, captan, chinomethionat, dazomet, debacarb, diclomezine, difenzoquat, difenzoquat-metilsulfate, fenoxanil, folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxypyrimidin-2-yl)-2-methyl-1H-benzimidazole, 5-chloro-7-(4-methylpiperidine-1-yl)-6-(2,4,6-trifluorophenyl)-[1,2,4]triazolo[1,5-a]pyrimidine, 6-(3,4-dichlorophenyl)-5-methyl-[1,2,4]triazolo[1,5-a]pyrimidin-7-ylamine, 6-(4-tert-butylphenyl)-5-methyl-[1,2,4]triazolo[1,5-a]pyrimidin-7-ylamine, 5-methyl-6-(3,5,5-trimethylhexyl)-[1,2,4]triazolo[1,5-a]pyrimidin-7-ylamine, 5-methyl-6-octyl-[1,2,4]triazolo[1,5-a]pyrimidin-7-ylamine, 6-methyl-5-octyl-[1,2,4]triazolo[1,5-a]pyrimidin-7-ylamine, 6-ethyl-5-octyl-[1,2,4]triazolo[1,5-a]pyrimidin-7-ylamine, 5-ethyl-6-octyl-[1,2,4]triazolo[1,5-a]pyrimidin-7-ylamine, 5-ethyl-6-(3,5,5-trimethylhexyl)-[1,2,4]triazolo[1,5-a]pyrimidin-7-ylamine, 6-octyl-5-propyl-[1,2,4]triazolo[1,5-a]pyrimidin-7-ylamine, 5-methoxymethyl-6-octyl-[1,2,4]triazolo[1,5-a]pyrimidin-7-ylamine, 6-octyl-5-trifluoromethyl-[1,2,4]triazolo[1,5-a]pyrimidin-7-ylamine and 5-trifluoromethyl-6-(3,5,5-trimethylhexyl)-[1,2,4]triazolo[1,5-a]pyrimidin-7-ylamine;

E) Carbamates and dithiocarbamates
thio- and dithiocarbamates: ferbam, mancozeb, maneb, metam, methasulfocarb, metiram, propineb, thiram, zineb, ziram;
carbamates: diethofencarb, benthiavalicarb, iprovalicarb, propamocarb, propamocarb hydrochloride, valiphenal, 4-fluorophenyl N-(1-(1-(4-cyanophenyl)ethylsulfonyl)but-2-yl)carbamate;

F) Other fungicides
guanidines: dodine, dodine free base, guazatine, guazatine acetate, iminoctadine, iminoctadine triacetate, iminoctadine trialbesilate;
antibiotics: kasugamycin, kasugamycin hydrochloride hydrate, polyoxins, streptomycin, validamycin A;
nitrophenyl derivatives: binapacryl, dicloran, dinobuton, dinocap, nitrothal-isopropyl, tecnazene;
organometallic compounds: fentin salts, such as, for example, fentin acetate, fentin chloride, fentin hydroxide;
sulfur-comprising heterocyclyl compounds: dithianon, isoprothiolane;
organophosphorus compounds: edifenphos, fosetyl, fosetyl-aluminum, iprobenfos, phosphorous acid and its salts, pyrazophos, tolclofos-methyl;
organochlorine compounds: chlorthalonil, dichlofluanid, dichlorophen, flusulfamide, hexachlorobenzene, pencycuron, pentachlorophenol and its salts, phthalide, quintozene, thiophanate-methyl, tolylfluanid, N-(4-chloro-2-nitrophenyl)-N-ethyl-4-methylbenzenesulfonamide;
inorganic active compounds: phosphorous acid and its salts, Bordeaux mixture, copper salts, such as, for example, copper acetate, copper hydroxide, copper oxychloride, basic copper sulfate, sulfur;
others: biphenyl, bronopol, cyflufenamid, cymoxanil, diphenylamine, metrafenone, mildiomycin, oxine-copper, prohexadione-calcium, spiroxamine, tolylfluanid, N-(cyclopropylmethoxyimino(6-difluoromethoxy-2,3-difluorophenyl)methyl)-2-phenylacetamide, N′-(4-(4-chloro-3-trifluoromethylphenoxy)-2,5-dimethylphenyl)-N-ethyl-N-methylformamidine, N′-(4-(4-fluoro-3-trifluoromethyl)phenoxy)-2,5-dimethylphenyl)-N-ethyl-N-methylformamidine, N′-(2-methyl-5-trifluoromethyl-4-(3-(trimethylsilanyl)propoxy)phenyl)-N-ethyl-N-methylformamidine, N′-(5-difluoromethyl-2-methyl-4-(3-trimethylsilanyl)propoxy)phenyl)-N-ethyl-N-methylformamidine, 2-{1-[2-(5-methyl-3-trifluoromethyl)pyrazole-1-yl)pacetyl]-piperidin-4-yl}thiazole-4-carboxylic acid methyl(1,2,3,4-tetrahydronaphth-1-yl)amide, 2-{1-[2-(5-methyl-3-(trifluoromethyl)pyrazol-1-yl)acetyl]piperidin-4-yl}thiazole-4-carboxylic acid methyl(R)-1,2,3,4-tetrahydronaphth-1-yl)amide, 6-(tert-butyl)-8-fluoro-2,3-dimethylquinolin-4-yl acetate, 6-(tert-butyl)-8-fluoro-2,3-dimethylquinolin-4-yl methoxyacetate.

Use may be made, as growth regulator, for example, of: abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol , fluthiacet, forchlorfenuron, gibberellic acid, inabenfide, indol-3-acetic acid, maleic hydrazide, mefluidide, mepiquat (mepiquat chloride), metconazole, naphthylacetic acid, N⁶-benzyladenine, paclobutrazol, prohexadione (prohexadione-calcium), prohydrojasmon, thidiazuron, triapenthenol, tributyl phosphorotrithioate, 2,3,5-triiodobenzoic acid, trinexapac-ethyl and uniconazole.

Use may be made, as herbicide, for example, of:
acetamides: acetochlor, alachlor, butachlor, dimethachlor, dimethenamid, flufenacet, mefenacet, metolachlor, metazachlor, napropamide, naproanilide, pethoxamid, pretilachlor, propachlor, thenylchlor;
amino acid analogs: bilanafos, glyphosate, glufosinate, sulfosate;
aryloxyphenoxypropionates: clodinafop, cyhalofop-butyl, fenoxaprop, fluazifop, haloxyfop, metamifop, propaquizafop, quizalofop, quizalofop-P-tefuryl;
bipyridyls: diquat, paraquat;
carbamates and thiocarbamates: asulam, butylate, carbetamide, desmedipham, dimepiperate, eptam (EPTC), esprocarb, molinate, orbencarb, phenmedipham, prosulfocarb, pyributicarb, thiobencarb, triallate;
cyclohexanediones: butroxydim, clethodim, cycloxydim, profoxydim, sethoxydim, tepraloxydim, tralkoxydim;
dinitroanilines: benfluralin, ethalfiuralin, oryzalin, pendimethalin, prodiamine, trifluralin;
diphenyl ethers: acifluorfen, aclonifen, bifenox, diclofop, ethoxyfen, fomesafen, lactofen, oxyfluorfen;
hydroxybenzonitriles: bromoxynil, dichlobenil, ioxynil;
imidazolinones: imazamethabenz, imazamox, imazapic, imazapyr, imazaquin, imazethapyr;
phenoxyacetic acids: clomeprop, 2,4-dichlorophenoxyacetic acid (2,4-D), 2,4-DB, dichlorprop, MCPA, MCPA-thioethyl, MCPB, mecoprop;
pyrazines: chloridazon, flufenpyr-ethyl, fluthiacet, norflurazon, pyridate;
pyridines: aminopyralid, clopyralid, diflufenican, dithiopyr, fluridone, fluroxypyr, picloram, picolinafen, thiazopyr;
sulfonylureas: amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propylimidazo[1,2-b]pyridazin-3-yl)sulfonyl)-3-(4,6-dimethoxypyrimidin-2-yl)urea;
triazines: ametryn, atrazine, cyanazine, dimethametryn, ethiozin, hexazinone, metamitron, metribuzin, prometryn, simazine, terbuthylazine, terbutryn, triaziflam;
ureas: chlorotoluron, daimuron, diuron, fluometuron, isoproturon, linuron, methabenzthiazuron, tebuthiuron;
other acetolactate synthase inhibitors: bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, pyroxsulam;
others: amicarbazon, aminotriazole, anilofos, beflubutamid, benazolin, benzocarbazone, benfuresate, benzofenap, bentazone, benzobicyclon, bromacil, bromobutide, butafenacil, butamifos, cafenstrole, carfentrazone, cinidon-ethyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, fluorochloridone, flurtamone, indanofan, isoxaben, isoxaflutole, lenacil, propanil, propyzamide, quinclorac, quinmerac, mesotrione, methylarsenic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil, sulcotrione, sulfentrazone, terbacil, tefuryltrione, tembotrione, thiencarbazone, topramezone, 4-hydroxy-3-[2-(2-methoxyethoxymethyl)-6-(trifluoromethyl)pyridin-3-carbonyl]bicyclo[3.2.1]oct-3-en-2-one, ethyl (3-[2-chloro-4-fluoro-5-(3-methyl-2,6-dioxo-4-trifluoromethyl-3,6-dihydro-2H-pyrimidin-1-yl)phenoxy]pyridin-2-yloxy)acetate, 6-amino-5-chloro-2-cyclopropyl-pyrimidine-4-carboxylate, 6-chloro-3-(2-cyclopropyl-6-methylphenoxy)pyridazin-4-ol, 4-amino-3-chloro-6-(4-chlorophenyl)-5-fluoropyridine-2-carboxylic acid, methyl 4-amino-3-chloro-6-(4-chloro-2-fluoro-3-methoxyphenyl)pyridine-2-carboxylate and methyl 4-amino-3-chloro-6-(4-chloro-3-dimethylamino-2-fluorophenyl)pyridine-2-carboxylate.

Use may be made, as insecticide, for example, of:
organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprofos, tetrachlorvinphos, terbufos, triazophos, trichlorfon;
carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;
insect growth inhibitors: a) chitin synthesis inhibitors: benzoylureas: chlorfluazuron, cyromazine, diflubenzuron, flucycloxuron, flufenoxuron, hexaflumuron, lufenuron, novaluron, teflubenzuron, triflumuron, buprofezin, diofenolan, hexythiazox, etoxazole, clofentezine; b) ecdysone antagonists: halofenozide, methoxyfenozide, tebufenozide, azadirachtin; c) juvenile hormone mimics: pyriproxyfen, methoprene, fenoxycarb; d) lipid biosynthesis inhibitors: spirodiclofen, spiromesifen, spirotetramat;
nicotine receptor agonists/antagonists: clothianidin, dinotefuran, imidacloprid, thiamethoxam, nitenpyram, acetamiprid, thiacloprid, 1-(2-chlorothiazol-5-ylmethyl)-2-nitrimino-3,5-dimethyl-1,3,5-triazinane;
GABA antagonists: endosulfan, ethiprole, fipronil, vaniliprole, pyrafluprole, pyriprole, 5-amino-1-(2,6-dichloro-4-methylphenyl)-4-sulfinamoyl-1H-pyrazole-3-thiocarboxamide;
macrocyclic lactones: abamectin, emamectin, milbemectin, lepimectin, spinosad, spinetoram;
mitochondrial electron transport inhibitor (METI) I acaricides: fenazaquin, pyridaben, tebufenpyrad, tolfenpyrad, flufenerim;
METI II and III substances: acequinocyl, fluacrypyrim, hydramethylnon;
uncouplers: chlorfenapyr;
inhibitors of oxidative phosphorylation: cyhexatin, diafenthiuron, fenbutatin oxide, propargite;
insect molting inhibitors: cyromazine;
inhibitors of mixed function oxidases: piperonyl butoxide;
sodium channel blockers: indoxacarb, metaflumizone;
others: benclothiaz, bifenazate, cartap, flonicamid, pyridalyl, pymetrozine, sulfur, thiocyclam, flubendiamide, chlorantraniliprole, cyazypyr (HGW86), cyenopyrafen, flupyrazofos, cyflumetofen, amidoflumet, imicyafos, bistrifluron and pyrifluquinazon.

In a preferred embodiment, the plant protection agents are preferably herbicides. In an additional preferred embodiment the plant protection agents are preferably insecticides. In an additional preferred embodiment the plant protection agents are preferably fungicides. In an additional preferred embodiment, the fungicides are preferably azoles. In an additional preferred embodiment the azoles are preferably triticonazole, epoxiconazole, fluquinconazole or metconazole.

Agrochemical adjuvants are additional suitable effect compounds. Adjuvants are compounds or mixtures of compounds which have no pesticidal effects on their own but increase the effectiveness of a pesticide. Penetration promoters are examples. All those substances which are conventionally used to improve the penetration of agrochemical active compounds into plants are suitable as penetration promoters. Penetration promoters are in this connection defined in this way, that they penetrate from an aqueous spray mixture and/or from the spray coating into the cuticle of a plant and, through this, can increase the mobility of active compounds in the cuticle. The method of unilateral desorption described in the literature (Baur et al., 1997, Pesticide Science 51, 131-152) can be used to determine this property. An additional suitable method consists in leaving, on a leaf, an individual drop of the mixture to be investigated and determining the residue on the leaf after several days.

Additives for food or animal fodder, such as food colorants, amino acids, vitamins, preservatives, antioxidants, fragrances or flavors, are additional suitable effect compounds.

Examples of auxiliaries for polymers are flame retardants, viscosity modifiers or polar liquids, such as can be used in the discontinuous phase. Examples of auxiliaries for paper are alkenylsuccinic anhydrides or dialkyldiketenes. Examples of auxiliaries for detergents and cleaners are surfactants or emulsifiers, such as can also be used as dispersants in the inverse miniemulsion. Enzymes such as hydrolases or amidases, can likewise be used as auxiliaries.

Biocides, plant protection agents and fertilizers are preferred effect compounds. In one embodiment, the effect compounds are plant protection agents. In an additional embodiment, the effect compounds are biocides. In an additional embodiment, the effect compounds are agrochemical adjuvants.

The effect compounds can be used in pure form, industrial rate, as extract or in a mixture with other effect compounds. The effect compounds are present dissolved or in a solid form in the dispersed phase. The total amount of the effect compounds is from 0.1 to 90% by weight, preferably from 5 to 50% by weight, based on the total charge in stage A).

The effect compounds can be released from the microparticles by means of diffusion from the microparticle or by disintegration of the microparticle. The rate of release can be selectively controlled through internal and external influences which affect the diffusion or the disintegration.

Additional additives, for example preservatives, thickeners, separating agents or protective colloids and emulsifiers, such as can also be used in the process according to the invention, are known to a person skilled in the art and are added in the usual amounts depending upon the use intended after the manufacture of the microparticles.

The wall monomers are chosen from the group consisting of ethylenically unsaturated monomers, polyisocyanates and/or polyepoxides. The polyisocyanates are preferably used in combination together with an additional wall monomer, such as ethylenically unsaturated monomers and polyisocyanates, polyisocyanates and polyols, or polyisocyanates and polyamines. Preference is given, as wall monomers, to ethylenically unsaturated monomers, ethylenically unsaturated monomers and polyisocyanates, ethylenically mono- and polyunsaturated monomers, polyisocyanates and polyols, polyisocyanates and polyamines, and polyepoxides and polyamines. Particular preference is given, as wall monomers, to ethylenically unsaturated monomers, and ethylenically unsaturated monomers and polyisocyanates. Special preference is given, as wall monomers, to ethylenically unsaturated monomers.

Suitable ethylenically unsaturated monomers are radically polymerizable monomers with at least one, preferably one, carbon-carbon double bond. Preferred ethylenically unsaturated monomers are (meth)acrylic acid, (meth)acrylates, (meth)acrylamide or vinyllactams, in particular (meth)acrylic acid, (meth)acrylates, or (meth)acrylamide. Mention may be made, as examples, of: acrylic acid and its esters, methacrylic acid and its esters, maleic acid and its esters, styrene, butadiene, isoprene, vinyl acetate, vinyl propionate, vinylpyridine, vinyl chloride, vinylidene dichloride, acrylonitrile, methacrylamide, itaconic acid, maleic anhydride, N-vinylpyrrolidone, acrylamido-2-methylpropanesulfonic acid, N-methylolacrylamide, N-methylolmethacrylamide, dimethylaminoethyl methacrylate and diethylaminoethyl methacrylate. Preferred esters of acrylic acid or methacrylic acid are C₁-C₂₄-alkyl esters, in particular hydroxyfunctional alkyl esters, especially hydroxyfunctional C₂-C₆-alkyl esters, such as 2-hydroxyethyl acrylate, 2-hydroxyethyl methacrylate, 4-hydroxybutyl acrylate, 4-hydroxybutyl methacrylate, 2-hydroxypropyl acrylate, 2-hydroxypropyl methacrylate, 3-hydroxypropyl acrylate, 3-hydroxypropyl methacrylate.

In a preferred embodiment, water-soluble ethylenically unsaturated monomers with a solubility in water of at least 5% by weight are suitable. Examples are acrylamide, methacrylamide, acrylic acid, methacrylic acid, salts of acrylamido-2-methylpropanesulfonic acid, 2-hydroxyethyl acrylate, 2-hydroxyethyl methacrylate, 4-hydroxybutyl acrylate, 4-hydroxybutyl methacrylate, 2-hydroxypropyl acrylate, 2-hydroxypropyl methacrylate, 3-hydroxypropyl acrylate or 3-hydroxypropyl methacrylate. Ethylenically unsaturated monomers which are very particularly preferred are hydroxyfunctional C₂-C₆-alkyl esters of acrylic acid or methacrylic acid, and vinylpyrrolidone, very specially 2-hydroxyethyl acrylate, 2-hydroxyethyl methacrylate, 4-hydroxybutyl acrylate, 4-hydroxybutyl methacrylate, 2-hydroxypropyl acrylate, 2-hydroxypropyl methacrylate, 3-hydroxypropyl acrylate or 3-hydroxypropyl methacrylate.

Suitable polyisocyanates are aliphatic and aromatic isocyanates with at least two, preferably from two to four, particularly preferably from two to three isocyanate groups. Examples of polyisocyanates are aromatic isocyanates such as 2,4-tolylene diisocyanate (2,4-TDI), 2,4′-diphenylmethane diisocyanate (2,4′-MDI) and “TDI-mixtures” (mixtures of 2,4-tolylene diisocyanate and 2,6-tolylene diisocyanate). Mention may be made, as aliphatic isocyanates, for example, of: 1,4-butylene diisocyanate, hexamethylene diisocyanate (HDI), 1,12-dodecamethylene diisocyanate, 1,10-decamethylene diisocyanate, 2-butyl-2-ethylpentamethylene diisocyanate, 2,4,4- or 2,2,4-trimethylhexamethylene diisocyanate, isophorone diisocyanate (IPDI), 2-isocyanatopropylcyclohexyl isocyanate, 2,4′-methylenebiscyclohexyl diisocyanate and 4-methylcyclohexane 1,3-diisocyanate (H-TDI). Polyisocyanates which are suitable furthermore are oligoisocyanates and their mixtures. With oligoisocyanates, the number of the isocyanate groups is determined, usually through the NCO content, and the mean number of the isocyanate groups is calculated from that. This mean number of the isocyanate groups is typically at least two, preferably two to four, particularly preferably from two to three. Preferred oligoisocyanates are based on the abovementioned aromatic and/or aliphatic polyisocyanates, especially on diphenylmethane diisocyanate and/or hexamethylene diisocyanate. Such oligoisocyanates are, for example, commercially available as Lupranat® M20S from BASF SE. Preferred polyisocyanates are tolylene diisocyanate (2,4-TDI), 2,4′-diphenylmethane diisocyanate (2,4′-MDI), hexamethylene diisocyanate (HDI), isophorone diisocyanate (IPDI), and oligoisocyanates. Oligoisocyanates are particularly preferred.

The polyisocyanates can be prepared in the absence or, preferably in the presence of at least one polyurethane catalyst. All catalysts conventionally used in polyurethane chemistry, for example, are suitable as polyurethane catalysts, such as organic amines, in particular tertiary aliphatic, cycloaliphatic or aromatic amines, and Lewis acid organic metal compounds. Tin compounds, e.g., are possible as Lewis acid organic metal compounds, such as, for example, tin(II) salts of organic carboxylic acids, e.g. tin(II) acetate, tin(II) octoate, tin(II) ethylhexanoate and tin(II) laurate and the dialkyltin(IV) derivatives of organic carboxylic acids, e.g. dimethyltin diacetate, dibutyltin diacetate, dibutyltin dibutyrate, dibutyltin bis(2-ethylhexanoate), dibutyltin dilaurate, dibutyltin maleate, dioctyltin dilaurate and dioctyltin diacetate. Metal complexes, such as iron, titanium, zinc, aluminum, zirconium, manganese, nickel and cobalt acetylacetonates, are also possible.

Suitable polyepoxides are compounds with at least two, preferably from two to three epoxide groups. Examples of these are epoxides derived from bisphenol A, such as bisphenol A diglycidyl ether, or epoxides of the epichlorohydrin-substituted bis- or polyphenol type (epoxides with a degree of polymerization of from 1 to 2, commercially available under the description Epikote® E 828 from Shell) or tetraglycidylmethylenedianiline (e.g. LY 1802 from Ciba).

Furthermore, combinations comprising ethylenically unsaturated monomers and polyisocyanates are preferred as wall monomers. Suitable ethylenically unsaturated monomers and polyisocyanates are described above. Preferred ethylenically monounsaturated monomers for combination with polyisocyanates are hydroxyfunctional ethylenically unsaturated monomers, such as hydroxyfunctional C₂-C₆-alkyl esters of acrylic acid or methacrylic acid, especially 2-hydroxyethyl acrylate, 2-hydroxyethyl methacrylate, 4-hydroxybutyl acrylate, 4-hydroxybutyl methacrylate, 2-hydroxypropyl acrylate, 2-hydroxypropyl methacrylate, 3-hydroxypropyl acrylate or 3-hydroxypropyl methacrylate.

Furthermore, combinations comprising ethylenically mono- and polyunsaturated monomers are preferred as wall monomers. The term “ethylenically monounsaturated monomers” is to be understood as meaning monomers with exactly one radically polymerizable carbon-carbon double bond. The term “ethylenically polyunsaturated monomers” is understood to mean monomers with at least two, preferably from two to three, in particular two, radically polymerizable carbon-carbon double bonds which are preferably non-conjugated.

Suitable ethylenically monounsaturated monomers are listed above in the description of ethylenically unsaturated monomers. Preferred ethylenically monounsaturated monomers are hydroxyfunctional C₂-C₆-alkyl esters of acrylic acid or methacrylic acid, and vinylpyrrolidone, very specially 2-hydroxyethyl acrylate, 2-hydroxyethyl methacrylate, 4-hydroxybutyl acrylate, 4-hydroxybutyl methacrylate, 2-hydroxypropyl acrylate, 2-hydroxypropyl methacrylate, 3-hydroxypropyl acrylate or 3-hydroxypropyl methacrylate.

Suitable ethylenically polyunsaturated monomers are the diesters of diols with acrylic acid or methacrylic acid, and furthermore the diallyl and divinyl ethers of these diols. Mention may be made, by way of example, of ethanediol diacrylate, ethylene glycol dimethacrylate, polyalkylene glycol di(meth)acrylate, generally ethylene and/or propylene being used as alkylene, 1,3-butylene glycol dimethacrylate, methallylmethacrylamide, allyl acrylate and allyl methacrylate. Also suitable are divinylbenzene, trivinylbenzene and divinylcyclohexane and trivinylcyclohexane, polyesters of polyols with acrylic acid and/or methacrylic acid, and furthermore the polyallyl and polyvinyl ethers of these polyols. Preference is given to propanediol, butanediol, pentanediol and hexanediol diacrylates and the corresponding methacrylates, trimethylolpropane triacrylate and trimethacrylate, pentaerythrityl triallyl ether, pentaerythrityl tetraallyl ether, pentaerythrityl triacrylate and pentaerythrityl tetraacrylate or the corresponding methacrylates, and also their industrial mixtures. Particular preference is given to propanediol, butanediol, pentanediol and hexanediol diacrylates and the corresponding methacrylates.

Preferred combinations comprising ethylenically mono- and polyunsaturated monomers are 2-hydroxyethyl (meth)acrylate and pentaerythrityl triacrylate; 2-hydroxyethyl (meth)acrylate and butanediol di(meth)acrylate; and 2-hydroxyethyl (meth)acrylate and polyalkylene glycol di(meth)acrylate.

Furthermore, combinations comprising polyisocyanates and polyols are preferred as wall monomers. Suitable polyisocyanates have been described above. Suitable as polyols are alcohols with at least two alcohol groups, such as ethanediol, diethylene glycol, 1,2- or 1,3-propanediol, dipropylene glycol, 1,4-butanediol, 1,5-pentanediol, 1,6-hexanediol, 1,10-decanediol, glycerol and trimethylolpropane, and furthermore also dialcohols, which comprise aromatic or aliphatic ring systems, such as e.g., 1,4-bisdihydroxymethylbenzene or 1,4-bisdihydroxyethylbenzene. Use may furthermore be made of polyesterpolyols from lactones, e.g. caprolactone, or hydroxycarboxylic acids, e.g. hydroxycaproic acid. Polymers with at least two alcohol groups can likewise be used, such as polyvinyl alcohol or partially hydrolyzed polyvinyl acetate. Mixtures of the abovementioned polyols are likewise possible. Preferred polyalcohols are diethylene glycol, 1,4-butanediol, 1,5-pentanediol and 1,6-hexanediol.

Furthermore, combinations comprising polyisocyanates and polyamines are preferred as wall monomers. Suitable polyisocyanates have been described above. Compounds with at least two, preferably from two to four, in particular from two to three, amino groups can be used as polyamines. Possible polyamines are preferably primary and secondary aliphatic polyamines. Mention may be made, for example, of: 1,2-ethylenediamine, diethylenetriamine, triethylenetetramine, bis(3-aminopropyl)amine, bis(2-methylaminoethyl)methylamine, 1,4-diaminocyclo-hexane, 3-aminomethylaminopropane, N-methyl-bis(3-aminopropyl)amine, 1,4-diamino-n-butane, 1,6-diamino-n-hexane, polyvinylamine or amino-terminated polyether. Preferred polyamines are 1,2-ethylenediamine, diethylenetriamine and triethylenetetramine.

Furthermore, combinations comprising polyepoxides and polyamines are preferred as wall monomers. Suitable polyepoxides and polyamines have been described above.

The wall monomers are generally used in a wall monomer to polyester monomer ratio by weight of 1:5 to 10:1, preferably 1:3 to 7:1, particularly preferably 1:1 to 4:1.

The process according to the invention for the preparation of effect compound-comprising microparticles M comprises A) the formation of a crude suspension of microparticles A by means of enzymatic polyester synthesis in an inverse miniemulsion comprising enzyme, effect compound and polyester monomers; and B) the polymerization of wall monomers from the group consisting of ethylenically unsaturated monomers, polyisocyanates and/or polyepoxides in the crude suspension of microparticles A. Stages A) and B) are usually carried out in the order mentioned.

In stage A), the crude suspension of microparticles A is formed by means of enzymatic polyester synthesis in an inverse miniemulsion comprising enzyme, effect compound and polyester monomers. Generally, in this connection, at least one dispersant, at least one nonpolar liquid, at least one polar liquid, at least one polyester monomer, at least one enzyme which catalyzes the polymerization and at least one effect compound are brought together in any order and an inverse miniemulsion is prepared therefrom. It is likewise possible to prepare premixes of individual components. Preferably, at least one enzyme which catalyzes the polymerization of the polyester monomer is introduced in a preprepared inverse miniemulsion.

The process according to the invention is preferably carried out in such a way that at least one dispersant is introduced into at least a portion of a liquid and a portion of the polyester monomers. The effect compound and a portion of the polyester monomers are introduced separately therefrom into at least a portion of the liquid. The two mixtures are brought together and an inverse miniemulsion is prepared. Subsequently, portions of the polyester monomers and also the enzyme are introduced into the miniemulsion. The term “portion of the polyester monomers” means, in this connection, between 0 to 100% of the total polyester monomers present in the reaction charge. The term “at least a portion” means more than 0% of the amount present in the total charge. In a preferred embodiment, a portion of the polyester monomers is introduced into the miniemulsion, the portion being more than 1%, preferably more than 10%.

Additional additives, such as preservatives, can be introduced in any processing stage.

The process according to the invention is generally carried out at a reaction temperature of 5 to 100° C., often of 20 to 80° C. and frequently of 30 to 65° C. Generally, the process is carried out at a pressure (absolute value) generally from 0.8 to 10 bar, preferably from 0.9 to 2 bar and in particular at 1 bar (atmospheric pressure). A person skilled in the art suits the reaction time to the desired properties of the microparticles, for example the degree of polymerization. After the desired reaction time, the enzyme can be destroyed or reused, the microparticles can be isolated or the reaction mixture can be otherwise isolated or reprocessed. Preferably, the crude suspension of microparticles A is used directly for stage B).

The inverse miniemulsion which has to be present according to the invention can be prepared according to the prior art. For this, a macroemulsion is prepared by introducing energy into the mixture of the phases by shaking, beating, stirring or turbulent mixing; by injecting one liquid into another; by vibrations and cavitation in the mixture (e.g. ultrasound); through emulsifying centrifuges; through colloid mills and homogenizers; or by means of a discharge nozzle, such as described, for example, in WO 2006/053712. The macroemulsion is converted by homogenization into a miniemulsion with droplet sizes of less than 1000 nm. The homogenization is preferably carried out at 0 to 100° C. by application of ultrasound, high pressure homogenizers or other high-energy homogenizing devices, such as discharge nozzles.

Generally, solid microparticles are formed from the polyester monomers during the reaction time in the inverse miniemulsion under catalysis of the enzyme. Through the formation of solid microparticles, a crude suspension of microparticles A results from the inverse miniemulsion.

In stage B), wall monomers from the group consisting of ethylenically unsaturated monomers, polyisocyanates and/or polyepoxides are polymerized in the crude suspension of microparticles A. Preferably, at least one wall monomer is introduced into a preprepared crude suspension of microparticles A and subsequently polymerized. Particularly preferably, at least one wall monomer and at least one dispersant are added to the crude suspension. The wall monomers can be polymerized conventionally, such as through polymerization catalysts or physical methods. If the wall monomers comprise ethylenically unsaturated monomers, then generally radical initiators are added as polymerization catalysts and/or the reaction temperature is increased. If the wall monomers comprise polyisocyanates, then generally the abovementioned polyurethane catalysts are added as polymerization catalysts.

Stage B) is preferably carried out in such a way that the crude suspension from stage A) is treated with at least one dispersant and at least one wall monomer. Preferably, the crude suspension is treated with an emulsion comprising wall monomer and dispersant. Preferably, in this connection, an emulsion of wall monomers is formed in the crude suspension of microparticles A. Subsequently, at least one polymerization catalyst is added. Wall monomer, dispersant and polymerization catalyst can be added in one amount, in several portions or continuously. Wall monomer, dispersant and polymerization catalyst can be dissolved or dispersed in polar or nonpolar solvent before they are added to the crude suspension.

In an additional preferred embodiment, at least one wall monomer is already added in stage A) and only polymerized during stage B). Preferably, use is made for this purpose of wall monomers which do not carry any primary or secondary hydroxyl groups. Ethylenically unsaturated monomers which do not carry any primary or secondary hydroxyl groups are particularly suitable.

The process according to the invention is generally carried out at a reaction temperature of 20 to 120° C., often of 40 to 90° C. and frequently of 50 to 80° C. Generally, the process is carried out at a pressure (absolute values) generally of 0.8 to 10 bar, preferably of 0.9 to 2 bar and in particular at 1 bar (atmospheric pressure). A person skilled in the art suits the reaction time to the desired properties of the microparticles, for example the degree of polymerization. The reaction charge is usually mixed, for example, by continuous stirring.

Generally, in stage B) the effect compound comprising microparticles M are formed from the microparticles A and the polymerized wall monomers. In addition, in stage B), small proportions, preferably less than 20% by weight, in particular less than 5% by weight, based on the total amount of all microparticles, of microparticles not according to the invention, may be produced only from the polymerized wall monomers. This secondary nucleation is an extensive side reaction which a person skilled in the art can reduce by conventional measures, for example by slow metering of the wall monomers, or low concentration of the wall monomers in the continuous phase.

The further use of the microparticles M without further reprocessing is possible. After the preparation according to the invention of the microparticles, they can when required be isolated, that is be freed from solvents. Suitable methods are, for example, evaporation, spray drying, freeze drying, centrifuging, filtration or vacuum drying. In a preferred embodiment, the microparticles are not isolated after preparation.

Furthermore, the microparticles M can be converted to dispersions according to the invention in which the microparticles are dispersed in water or aqueous solutions, for example, by phase transfer processes or transfer processes analogous to flushing, or preferably, by drying the particles to give a powder which is subsequently redispersed.

The dispersion prepared according to the invention comprising microparticles M or the further reprocessed product can be used as component in colorants, cosmetics, drugs, plant protection agents, fertilizers, additives for food or animal fodder, or auxiliaries for polymers, paper, textiles, leather, coating materials, detergents or cleaners. It is advantageous for the effect compound to be able to be selectively re-released, in particular in the biosphere, where polyester-decomposing enzymes are ubiquitous.

In a preferred embodiment, the present invention relates to an agrochemical formulation comprising microparticles M according to the invention or microparticles M prepared according to the invention.

The agrochemical formulations can comprise additional formulating auxiliaries. The expression “formulating auxiliaries”, within the meaning of the invention, are auxiliaries which are suitable for the formulation of agrochemical active compounds, such as solvents, carriers, surfactants (ionic or nonionic surfactants, adjuvants, dispersants), preservatives, antifoaming agents and/or antifreeze agents. Auxiliaries for seed treatment can optionally also be dyes, binders, gelling agents and/or thickeners.

Generally, the agrochemical formulations can comprise from 0 to 90% by weight, preferably from 1 to 85% by weight, particularly preferably from 5 to 80% by weight and in particular from 5 to 65% by weight of formulating auxiliaries.

Furthermore, the present invention relates to a method for combating undesirable plant growth, wherein the undesirable plants, the soil in which the undesirable plants grow or their seeds are treated with an agrochemical formulation according to the invention.

In addition, the present invention relates to a method for combating undesirable insect or acarid infestation on plants and/or for combating phytopathogenic fungi, wherein the fungi/insects, their habitat or the plants or soil to be protected from fungal or insect infestation, or the plants, the soil in which the plants grow or their seeds, are treated with an agrochemical formulation according to the invention.

In addition, the present invention relates to a method for treating seed with an agrochemical formulation according to the invention and to seed treated with an agrochemical formulation according to the invention.

All in all, the process according to the invention has many advantages in comparison with conventional processes for the preparation of microparticles: lower reaction temperatures and largely neutral pHs allow the use of temperature- and pH-sensitive effect compounds; the polymers of the microparticle can be prepared directly in situ without carrying out expensive warehousing.

Likewise, the microparticles prepared according to the invention have advantages: the microparticles are denser than in other preparation processes. In particular, the microparticles are more stable mechanically than microparticles prepared only enzymatically. The microparticles can comprise temperature-labile or otherwise sensitive effect compounds; they can also comprise effect compounds dissolved in a polar liquid. Furthermore, the rate of release of the effect compound from the microparticles can be controlled with the type and/or amount of the wall monomers. The rate of release is advantageously slower through the polymerization of the wall monomers in comparison with particles which are formed only from polyester.

The following examples illustrate the invention without limiting it.

### EXAMPLES

Partially-hydrogenated petroleum distillate: partially hydrogenated petroleum distillate with boiling point of 260 to 280° C., for example, available commercially as Isopar® V from Exxon Mobil Chemical.

Enzyme: a lipase of Candida antarctica type B, immobilized on spherical polymer beads, for example, available commercially as Novozym® 435 from Novozymes, Denmark.

Dispersant: polyester/polyethylene oxide/polyester block copolymer with a molar mass >1000 g/mol, prepared by reaction of condensed 12-hydroxystearic acid with polyethylene oxide according to the teaching of EP 424 B1 (available commercially as Hypermer® B-246, from Croda).

Caprolactone: ε-caprolactone with purity >99%.

HEMA: 2-hydroxyethyl methacrylate, commercially available from BASF SE.

AIBN: azobisisobutyronitrile

DBTL: dibutyltin dilaurate

Isocyanate A: oligomeric 4,4′-diphenylmethane diisocyanate with NCO content of 31.8 g/100 g (ASTM D 5155-96 A), acidity of 150 mg/kg (as HCl, ASTM D 1638-74) and viscosity of 210 mPa.s (DIN 53018), for example, commercially available as Lupranat® M20S from BASF SE.

Use was made, as effect compound, of a fungicidal plant protection agent, for example, triticonazole. Alternatively, a colorant, for example, Basacid® Blue 756 (C.I. Acid Blue 9, triphenylmethane dye, for example, available from BASF SE), was used as effect compound. Basacid Blue 756 is insoluble in Isopar® V, while it dissolves in propylene carbonate and in caprolactone. As an additional alternative, propylene carbonate was used as effect compound.

The dye Sudan® Blue (anthraquinone dye, C.I. Solvent Blue 79, for example, available from BASF SE) was used for staining for light microscopy. It dissolves only in very hydrophobic media, such as in Isopar® V and polycaprolactone. However, it is sparingly soluble in water or propylene carbonate.

### Example 1

### Not According to the Invention

The following amounts were used for the preparation of the inverse miniemulsion:

120 g of partially hydrogenated petroleum distillate

24.0 g of propylene carbonate

6.0 g of ε-caprolactone

19.2 mg of D-sorbitol

1.65 g of triticonazole

3.0 g of dispersant

0.6 g of enzyme

The dispersant was introduced into a sample vessel and dissolved with stirring in partially hydrogenated petroleum distillate. Triticonazole and D-sorbitol were dissolved in an additional vessel in a mixture of caprolactone and propylene carbonate. The homogeneous solutions were then mixed with one another and pre-emulsified by stirring with a magnetic stirrer at ambient temperature for 60 min. An inverse miniemulsion was prepared from this using ultrasound (ultrasonic processor UP 400S from Hielscher) with cooling with an ice bath (5 min, 100% with Sonotrode H7) and, after addition of 100 mg of enzyme, polymerized at 60° C. for 48 h. A crude suspension of microparticles was obtained.

Intact spherical particles were revealed in light microscopy photographs (1000 times magnification) (FIG. 1).

For the preparation of an SEM (scanning electron microscope) photograph, the product obtained was centrifuged and the solid was washed with isobutanol and hexane and dried in the air. The SEM photograph at 5.00 kV showed largely decomposed microparticles (FIG. 2).

### Example 2

### Polymerization with 300% of Hydroxyethyl Methacrylate (HEMA)

The crude suspension of microparticles was first prepared as described in example 1. 3.6 g of dispersants were then added and the mixture was stirred for 15 min. After complete dissolution of the dispersant in the oil phase, 18.0 g of HEMA were added and the mixture stirred for a further 30 min. The polymerization reaction was subsequently initiated by addition of a mixture of 72 g of Isopar V and 0.36 g of AIBN. In order to guarantee complete conversion, the same amount of AIBN in 24 g Isopar V was again added after a reaction time at 60° C. of 6 h and polymerization was continued until conversion was complete.

For the preparation of an SEM (scanning electron microscope) photograph, the product thus obtained was, as in example 1, centrifuged and the solid thus obtained washed with isobutanol and hexane and dried in the air. The dried solid was subsequently finely ground in a mortar to give a powder. The SEM photograph showed intact spherical microparticles (FIG. 3).

For additional monitoring of the stability of the particles, the powder was redispersed in a 1% by weight aqueous SDS solution using ultrasound (1 min, with cooling using ice, 100% with Sonotrode H7). Intact spherical particles were revealed in light microscopy photographs (1000 times magnification).

The experimental studies reveal high mechanical stability, in particular in comparison with the particles from example 1.

### Example 3

### Polymerization with 200% of Hydroxyethyl Methacrylate

The crude suspension of microparticles was first prepared as described in example 1. 2.4 g of dispersants were then added and the mixture was stirred for 15 min. After complete dissolution of the dispersant in the oil phase, 12.0 g of HEMA were added and the mixture stirred for a further 30 min. The polymerization reaction was subsequently initiated by addition of a mixture of 24 g of partially hydrogenated petroleum distillate and 0.24 g of AIBN. In order to guarantee complete conversion, the same amount of AIBN in 24 g of partially hydrogenated petroleum distillate was again added after a reaction time at 60° C. of 6 h and polymerization was continued until conversion was complete. For the preparation of an SEM photograph, the product obtained was prepared as in example 2. The SEM photograph revealed intact, spherical microparticles.

### Example 4

### Polymerization with Three Times 100% of Hydroxyethyl Methacrylate

The crude suspension of microparticles was first prepared as described in example 1. 1.2 g of dispersant were then added and the mixture was stirred for 15 min. After complete dissolution of the dispersant in the oil phase, 6.0 g of HEMA were added and the mixture stirred for a further 30 min. The polymerization reaction was subsequently initiated by addition of a mixture of 24 g of partially hydrogenated petroleum distillate and 0.12 g of AIBN. After a reaction time at 60° C. of 6 h, the 6.0 g of HEMA were added and, after an additional 20 h, an additional 6.0 g of HEMA were added, in each case in combination with the addition of 1.2 g of dispersant and 0.12 g of AIBN in 24 g of Isopar V. After the last HEMA addition, polymerization was continued for 12 h at 60° C. until conversion was complete. For the preparation of an SEM photograph, the product obtained was prepared as in example 2. The SEM photograph revealed intact, spherical microparticles.

### Example 5

### Particles without Propylene Carbonate, Polymerization with 300% HEMA

The following amounts were used for the preparation of the inverse miniemulsion:

114.0 g of partially hydrogenated petroleum distillate

30.0 g of ε-caprolactone

96 mg of D-sorbitol

0.82 g of triticonazole

6.0 g of dispersant

3.0 g of Novozym 435

The dispersant was introduced into a sample vessel and dissolved with stirring in partially hydrogenated petroleum distillate. Triticonazole was dissolved in an additional vessel in a mixture of caprolactone and sorbitol. The homogeneous solutions were then mixed with one another and pre-emulsified by stirring with a magnetic stirrer (60 min at ambient temperature). An inverse miniemulsion was prepared from this using ultrasound (ultrasonic processor UP 400S from Hielscher) with cooling with an ice bath (5 min, 100% with Sonotrode H7) and, after addition of the enzyme, polymerized at 60° C. for 48 h.

93.0 g of the product obtained were subsequently treated with 10.8 g of dispersant and the mixture was stirred for 15 min. After complete dissolution of the dispersant in the oil phase, 54.0 g of HEMA were added and the mixture was stirred for a further 30 min. The polymerization reaction was subsequently initiated by addition of a mixture of 50 g of Isopar V and 1.1 g of AIBN. In order to guarantee complete conversion, the same amount of AIBN in 50 g of Isopar V was again added after reaction time at 60° C. of 6 h and polymerization was continued for 12 h until conversion was complete. For the preparation of an SEM photograph, the product obtained was prepared as in example 2. The SEM photograph revealed intact, spherical microparticles.

### Example 6

### Polymerization with 300% of HEMA and Isocyanate A

237.5 g of the final product obtained in example 5 were treated with stirring with 13.7 g of isocyanate A. After addition of DBTL as catalyst, the reaction mixture was stirred at 60° C. for 4 h until conversation was complete (reaction monitoring by FTIR). For the preparation of an SEM photograph, the product obtained was prepared as in example 2. The SEM photograph revealed intact, spherical microparticles.

### Example 7

### Polymerization with HEMA and Isocyanate A—NCO/OH ratio

In this example, OH groups of HEMA are crosslinked with isocyanate A at different ratios of OH to NCO.

30.0 g of the final product obtained in example 2 were treated with isocyanate A and heated to 60° C. while stirring with a magnetic stirrer. After addition of 0.01 g of DBTL as catalyst, the reaction mixture was stirred overnight until NCO conversion was complete. Amount of isocyanate A used:
a) 0.5 g (corresponds to NCO/OH=0.25)
b) 1.02 g (corresponds to NCO/OH=0.5)
c) 1.52 g (corresponds to NCO/OH=0.75)
d) 2.03 g (corresponds to NCO/OH=1.0)

For the preparation of an SEM photograph, the product obtained was in each case prepared as in example 2. The SEM photograph revealed intact, spherical microparticles.

1-14. (canceled)

15. A process for the preparation of effect compound-comprising microparticles M comprising:
A) forming a crude suspension of microparticles A by means of enzymatic polyester synthesis in an inverse miniemulsion comprising enzyme, effect compound and polyester monomers; and
B) polymerizing wall monomers from the group consisting of ethylenically unsaturated monomers, polyisocyanates and polyepoxides in the crude suspension of microparticles A.

16. The process of claim 15, wherein the wall monomer is selected from the group consisting of:
an ethylenically unsaturated monomer;
an ethylenically unsaturated monomer and/or a polyisocyanate;
an ethylenically mono- and/or polyunsaturated monomer;
a polyisocyanate and/or polyol;
a polyisocyanate and/or polyamine; and
a polyepoxides and a polyamine.

17. The process of claim 15, wherein the wall monomer is:
an ethylenically unsaturated monomer, or
an ethylenically unsaturated monomers and a polyisocyanate.

18. The process of claim 15, wherein the wall monomer is an ethylenically unsaturated monomer selected from (meth)acrylic acid, (meth)acrylate and (meth)acrylamide.

19. The process of claim 15, wherein the wall monomer is a polyisocyanateselected from aliphatic and aromatic polyisocyanates with a mean functionality of 2 to 3.5.

20. The process of claim 15, wherein the polyester monomer is a hydroxy acid.

21. The process of claim 15, wherein the wall monomers are used in a wall monomer to polyester monomer weight ratio of 1:5 to 10:1.

22. The process of claim 15, wherein the effect compound is a colorant, cosmetic, drug, biocide, plant protection agent, agrochemical adjuvant, fertilizer, additive for food or animal fodder, or auxiliary for polymers, paper, textiles, leather, detergents or cleaners.

23. The process of claim 22, wherein the effect compound is a plant protection agent or a fertilizer.

24. An effect compound-comprising microparticle M, obtained by means of the process of claim 15, wherein the effect compound is a plant protection agent or a mixture of plant protection agents.

25. An agrochemical formulation comprising the microparticle M of claim 24.

26. A method for combating undesirable plant growth, comprising treating the undesirable plants, the soil in which the undesirable plants grow or their seeds with the formulation of claim 25.

27. The method of claim 26, wherein the wall monomer is selected from the group consisting of:
an ethylenically unsaturated monomer;
an ethylenically unsaturated monomer and/or a polyisocyanate;
an ethylenically mono- and/or polyunsaturated monomer;
a polyisocyanate and/or polyol;
a polyisocyanate and/or polyamine; and
a polyepoxides and a polyamine.

28. The method of claim 26, wherein the wall monomer is:
an ethylenically unsaturated monomer, or
an ethylenically unsaturated monomers and a polyisocyanate.

29. The method of claim 26, wherein the wall monomer is an ethylenically unsaturated monomer selected from (meth)acrylic acid, (meth)acrylate and (meth)acrylamide.

30. The method of claim 26, wherein the wall monomer is a polyisocyanateselected from aliphatic and aromatic polyisocyanates with a mean functionality of 2 to 3.5.

31. The method of claim 26, wherein the polyester monomer is a hydroxy acid.

32. A method for combating undesirable insect or acarid infestation on plants and/or for combating phytopathogenic fungi, comprising treating the fungi/insects, their habitat or the plants or soil to be protected from fungal or insect infestation, or the plants, the soil in which the plants grow or their seeds, with the formulation of claim 25.

33. The method of claim 32, wherein the wall monomer is selected from the group consisting of:
an ethylenically unsaturated monomer;
an ethylenically unsaturated monomer and/or a polyisocyanate;
an ethylenically mono- and/or polyunsaturated monomer;
a polyisocyanate and/or polyol;
a polyisocyanate and/or polyamine; and
a polyepoxides and a polyamine.

34. A seed treated with the formulation of claim 25.
